(19) United States
(12) Patent Application Publication
(10) Pub. No.: US 2023/0263724 A1
(43) Pub. Date: Aug. 24, 2023

(54) Intravaginal Ring Device for the Delivery of Aromatase Inhibitor

(71) Applicant: Celanese EVA Performance Polymers LLC, Irving, TX (US)

(72) Inventors: Jeffrey Haley, Norwood, OH (US); Vijay Gyanani, Powell, OH (US); Karen Chen, Bound Brook, NJ (US); Thomas Quinci, Oxford, PA (US); Brian Wilson, Deerfield, IL (US)

(21) Appl. No.: 18/171,406

(22) Filed: Feb. 20, 2023

Related U.S. Application Data
(60) Provisional application No. 63/317,595, filed on Mar. 8, 2022.
(60) Provisional application No. 63/312,414, filed on Feb. 22, 2022.

(51) Int. Cl. A61K 9/00; A61K 31/4196; A61K 31/56; A61K 45/06

(52) U.S. Cl. CPC A61K 9/0036; A61K 31/4196; A61K 31/56; A61K 45/06

(57) ABSTRACT

An intravaginal ring device is provided. The core includes a core polymer matrix within which is dispersed a therapeutic agent comprising one or more aromatase inhibitors. The core polymer matrix contains an ethylene vinyl acetate copolymer. The ethylene vinyl acetate copolymer has a vinyl acetate content of from about 10 wt. % to about 60 wt. % and/or a melting temperature of from about 40° C. to about 120° C. as determined in accordance with ASTM D3418-15.

### RELATED APPLICATIONS

The present application is based upon and claims priority to U.S. Provisional Patent Application Ser. No. 63/312,414, having a filing date of Feb. 22, 2022 and U.S. Provisional Patent Application Ser. No. 63/317,595, having a filing date of Mar. 8, 2022, which are incorporated herein by reference.

### BACKGROUND

Each year, more than 180,000 women are diagnosed with breast cancer in the United States. If current breast cancer rates remain constant, each woman born today has a one-in-ten chance of developing breast cancer at a median onset age of about 60 to 65. Approximately two-thirds of post-menopausal breast cancer patients have estrogen-dependent disease thereby rendering them amenable to antiestrogen therapy. Estrogens promote growth and proliferation of specific target cells, such as breast epithelium and estrogen-dependent breast carcinoma cells. Thus, certain cancer therapies have focused on agents that block the estrogen receptor on tumor cells.

A variety of different drugs are used to treat estrogen-dependent cancers, such as aromatase inhibitors. Aromatase inhibitors, such as anastrozole, letrozole, and exemestane, block aromatase enzyme catalysis of androgens to estrogens thereby decreasing the amount of hormone ligand available to bind with the receptor. Accordingly, less estrogen is available to stimulate the growth of hormone-receptor positive breast cancer cells.

Treatment with aromatase inhibitors can be long lasting and can require treatment for many weeks, months, or even years. Patients taking a traditional oral dosage form may be required to take the oral dose at least once per day, if not multiple times per day, for the duration of treatment. For example, patients taking aromatase inhibitors can require an oral dose of medication at least twice a day for a year or longer. Such continuous dosage over a long period of time can result in decreased patient compliance, as many patients forget to take the medication or become tired of having to take many pills over an extended period of time.

As such, a need continues to exist for treatment options that are capable of delivering one or more aromatase inhibitors over a sustained period of time.

### BRIEF SUMMARY

In accordance with one embodiment of the present disclosure, an intravaginal ring device is disclosed. The device includes a core containing a core polymer matrix having one or more therapeutic agents including one or more aromatase inhibitors dispersed therein. The core polymer matrix contains an ethylene vinyl acetate copolymer. The ethylene vinyl acetate copolymer has a vinyl acetate content of from about 10 wt. % to about 60 wt. % and/or a melting temperature of from about 40° C. to about 120° C. as determined in accordance with ASTM D3418-15.

Other features and aspects of the present disclosure are set forth in greater detail below.

### BRIEF DESCRIPTION OF THE DRAWINGS

A full and enabling disclosure of the present disclosure, including the best mode thereof, directed to one of ordinary skill in the art, is set forth more particularly in the remainder of the specification, which makes reference to the appended drawings in which:

FIG. 1 is a perspective view of one embodiment of the intravaginal ring device of the present disclosure;

FIG. 2 is a cross-sectional view of the intravaginal ring device of FIG. 1;

FIG. 3 is a cross-sectional view of one embodiment of an intravaginal ring device of the present disclosure;

FIG. 4 is a cross-sectional view of one embodiment of an intravaginal ring device of the present disclosure;

FIG. 5 is a perspective view of another embodiment of the intravaginal ring device of the of the present disclosure;

FIG. 6 is a perspective view of another embodiment of the intravaginal ring device of the of the present disclosure;

FIG. 7 is a graph showing the cumulative release of exemestane per surface area versus time for Examples 1-5; and

FIG. 8 is a graph showing the cumulative release of exemestane per surface area versus time for Examples 6-11.

Repeat use of references characters in the present specification and drawing is intended to represent same or analogous features or elements of the disclosure.

### DETAILED DESCRIPTION

It is to be understood by one of ordinary skill in the art that the present discussion is a description of exemplary embodiments only, and is not intended as limiting the broader aspects of the present disclosure.

Generally speaking, the present disclosure is directed to an intravaginal ring device that is capable of delivering an aromatase inhibitor to a patient (e.g., human, pet, farm animal, racehorse, etc.) over a sustained period of time to help prohibit and/or treat a condition, disease, and/or cosmetic state of the patient. The condition and/or disease can include hormone receptor-positive early, locally advanced, and metastatic breast cancers or other pathological conditions where estrogen reduction is necessary. The intravaginal ring device includes a core containing a core polymer matrix containing an ethylene vinyl acetate copolymer having one or more therapeutic agents dispersed therein. The therapeutic agent includes one or more aromatase inhibitors. The ethylene vinyl acetate copolymer has a vinyl acetate content of from about 10 wt. % to about 60 wt. % and/or a melting temperature of from about 40° C. to about 120° C. as determined in accordance with ASTM D3418-15.

Various embodiments of the present disclosure will now be described in more detail.

### I. Core

As indicated above, the core polymer matrix contains at least a polymer that is generally hydrophobic in nature so that it can retain its structural integrity for a certain period of time when placed in an aqueous environment, such as the body of a mammal, and stable enough to be stored for an extended period before use. Examples of suitable hydrophobic polymers for this purpose may include, for instance, silicone polymer, polyolefins, polyvinyl chloride, polycarbonates, polysulphones, styrene acrylonitrile copolymers, polyurethanes, silicone polyether-urethanes, polycarbonate-urethanes, silicone polycarbonate-urethanes, etc., as well as combinations thereof. Of course, hydrophilic polymers that are coated or otherwise encapsulated with a hydrophobic polymer are also suitable for use in the core polymer matrix. Typically, the melt flow index of the hydrophobic polymer ranges from about 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-13 at a temperature of 190° C. and a load of 2.16 kilograms.

In certain embodiments, the core polymer matrix may contain a semi-crystalline olefin copolymer. The melting temperature of such an olefin copolymer may, for instance, range from about 40° C. to about 140° C., in some embodiments from about 50° C. to about 125° C., and in some embodiments, from about 60° C. to about 120° C., as determined in accordance with ASTM D3418-15. Such copolymers are generally derived from at least one olefin monomer (e.g., ethylene, propylene, etc.) and at least one polar monomer that is grafted onto the polymer backbone and/or incorporated as a constituent of the polymer (e.g., block or random copolymers). Suitable polar monomers include, for instance, a vinyl acetate, vinyl alcohol, maleic anhydride, maleic acid, (meth)acrylic acid (e.g., acrylic acid, methacrylic acid, etc.), (meth)acrylate (e.g., acrylate, methacrylate, ethyl acrylate, methyl methacrylate, ethyl methacrylate, etc.), and so forth. A wide variety of such copolymers may generally be employed in the polymer composition, such as ethylene vinyl acetate copolymers, ethylene (meth)acrylic acid polymers (e.g., ethylene acrylic acid copolymers and partially neutralized ionomers of these copolymers, ethylene methacrylic acid copolymers and partially neutralized ionomers of these copolymers, etc.), ethylene (meth)acrylate polymers (e.g., ethylene methylacrylate copolymers, ethylene ethyl acrylate copolymers, ethylene butyl acrylate copolymers, etc.), and so forth. Regardless of the particular monomers selected, certain aspects of the copolymer can be selectively controlled to help achieve the desired release properties. For instance, the polar monomeric content of the copolymer may be selectively controlled to be within a range of from about 10 wt. % to about 60 wt. %, in some embodiments about 20 wt. % to about 60 wt. %, and in some embodiments, from about 25 wt. % to about 50 wt. %. Conversely, the olefin monomeric content of the copolymer may be likewise within a range of from about 40 wt. % to about 90 wt. %, in some embodiments about 40 wt. % to about 80 wt. %, and in some embodiments, from about 50 wt. % to about 75 wt. %.

In one particular embodiment, for example, the core polymer matrix may contain at least one ethylene vinyl acetate polymer, which is a copolymer that is derived from at least one ethylene monomer and at least one vinyl acetate monomer. In certain cases, the present inventors have discovered that certain aspects of the copolymer can be selectively controlled to help achieve the desired release properties. For instance, the vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments from about 1 to about 200 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the core polymer matrix is from about 20 wt. % to about 90 wt. %, such as from about 30 wt. % to about 80 wt. %, such as from about 40 wt. % to about 70 wt. %.

Any of a variety of techniques may generally be used to form the ethylene vinyl acetate copolymer(s) with the desired properties as is known in the art. In one embodiment, the polymer is produced by copolymerizing an ethylene monomer and a vinyl acetate monomer in a high pressure reaction. Vinyl acetate may be produced from the oxidation of butane to yield acetic anhydride and acetaldehyde, which can react together to form ethylidene diacetate. Ethylidene diacetate can then be thermally decomposed in the presence of an acid catalyst to form the vinyl acetate monomer. Examples of suitable acid catalysts include aromatic sulfonic acids (e.g., benzene sulfonic acid, toluene sulfonic acid, ethylbenzene sulfonic acid, xylene sulfonic acid, and naphthalene sulfonic acid), sulfuric acid, and alkanesulfonic acids, such as described in U.S. Pat. No. 2,425,389 to Oxley et al.; U.S. Pat. No. 2,859,241 to Schnizer; and U.S. Pat. No. 4,843,170 to Isshiki et al. The vinyl acetate monomer can also be produced by reacting acetic anhydride with hydrogen in the presence of a catalyst instead of acetaldehyde. This process converts vinyl acetate directly from acetic anhydride and hydrogen without the need to produce ethylidene diacetate. In yet another embodiment, the vinyl acetate monomer can be produced from the reaction of acetaldehyde and a ketene in the presence of a suitable solid catalyst, such as a perfluorosulfonic acid resin or zeolite.

In certain embodiments, it may also be desirable to employ blends of an ethylene vinyl acetate copolymer and another hydrophobic polymer such that the overall blend and polymer matrix have a melting temperature and/or melt flow index within the range noted above. For example, the polymer matrix may contain a first ethylene vinyl acetate copolymer and a second ethylene vinyl acetate copolymer having a melting temperature that is greater than the melting temperature of the first copolymer. The second copolymer may likewise have a melt flow index that is the same, lower, or higher than the corresponding melt flow index of the first copolymer. The first copolymer may, for instance, have a melting temperature of from about 20° C. to about 60° C., in some embodiments from about 25° C. to about 55° C., and in some embodiments, from about 30° C. to about 50° C., such as determined in accordance with ASTM D3418-15, and/or a melt flow index of from about 40 to about 900 g/10 min, in some embodiments from about 50 to about 500 g/10 min, and in some embodiments, from about 55 to about 250 g/10 min, as determined in accordance with ASTM D1238-20 at a temperature of 190° C. and a load of 2.16 kilograms. The second copolymer may likewise have a melting temperature of from about 50° C. to about 100° C., in some embodiments from about 55° C. to about 90° C., and in some embodiments, from about 60° C. to about 80° C., such as determined in accordance with ASTM D3418-15, and/or a melt flow index of from about 0.2 to about 55 g/10 min, in some embodiments from about 0.5 to about 50 g/10 min, and in some embodiments, from about 1 to about 40 g/10 min, as determined in accordance with ASTM D1238-20 at a temperature of 190° C. and a load of 2.16 kilograms. The first copolymer may constitute from about 20 wt. % to about 80 wt. %, in some embodiments from about 30 wt. % to about 70 wt. %, and in some embodiments, from about 40 wt. % to about 60 wt. % of the polymer matrix, and the second copolymer may likewise constitute from about 20 wt. % to about 80 wt. %, in some embodiments from about 30 wt. % to about 70 wt. %, and in some embodiments, from about 40 wt. % to about 60 wt. % of the polymer matrix.

In certain cases, ethylene vinyl acetate copolymer(s) constitute the entire polymer content of the core polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix.

One or more therapeutic agents (e.g., aromatase inhibitors) are also dispersed within the core polymer matrix that are capable of prohibiting and/or treating a condition, disease, and/or cosmetic state a patient. The therapeutic agent may be prophylactically, therapeutically, and/or cosmetically active, systemically, or locally. The therapeutic agent can be homogenously dispersed within the core polymer matrix. Typically, therapeutic agents will constitute from about 5 wt. % to about 60 wt. %, in some embodiments from about 10 wt. % to about 50 wt. %, and in some embodiments, from about 15 wt. % to about 45 wt. % of the core, while the core polymer matrix constitutes from about 40 wt. % to about 95 wt. %, in some embodiments from about 50 wt. % to about 90 wt. %, and in some embodiments, from about 55 wt. % to about 85 wt. % of the core. Suitable therapeutic agents will be further discussed hereinbelow.

The core may also optionally contain one or more excipients if so desired, such as radiocontrast agents, release modifiers, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 20 wt. %, and in some embodiments, from about 0.05 wt. % to about 15 wt. %, and in some embodiments, from about 0.1 wt. % to about 10 wt. % of the core. In one embodiment, for instance, a radiocontrast agent may be employed to help ensure that the device can be detected in an X-ray based imaging technique (e.g., computed tomography, projectional radiography, fluoroscopy, etc.). Examples of such agents include, for instance, barium-based compounds, iodine-based compounds, zirconium-based compounds (e.g., zirconium dioxide), etc. One particular example of such an agent is barium sulfate. Other known antimicrobial agents and/or preservatives may also be employed to help prevent surface growth and attachment of bacteria, such as metal compounds (e.g., silver, copper, or zinc), metal salts, quaternary ammonium compounds, etc.

To help further control the release rate from the intravaginal ring device, a hydrophilic compound may also be incorporated into the core that is soluble and/or swellable in water. When employed, the weight ratio of the ethylene vinyl acetate copolymer(s) the hydrophilic compounds within the core may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Regardless of the particular components employed, the core may be formed through a variety of known techniques, such as by hot-melt extrusion, injection molding, solvent casting, dip coating, spray coating, microextrusion, coacervation, compression molding (e.g., vacuum compression molding), etc. In one embodiment, a hot-melt extrusion technique may be employed. Hot-melt extrusion is generally a solvent-free process in which the components of the core (e.g., hydrophobic polymer, therapeutic agent(s), optional excipients, etc.) may be melt blended and optionally shaped in a continuous manufacturing process to enable consistent output quality at high throughput rates. This technique is particularly well suited to various types of hydrophobic polymers, such as olefin copolymers. Namely, such copolymers typically exhibit a relatively high degree of long-chain branching with a broad molecular weight distribution. This combination of traits can lead to shear thinning of the copolymer during the extrusion process, which help facilitates hot-melt extrusion. Furthermore, the polar comonomer units (e.g., vinyl acetate) can serve as an “internal” plasticizer by inhibiting crystallization of the polyethylene chain segments. This may lead to a lower melting point of the olefin copolymer, which improves the overall flexibility of the resulting material and enhances its ability to be formed into devices of a wide variety of shapes and sizes.

During a hot-melt extrusion process, melt blending may occur at a temperature range of from about 20° C. to about 200° C., in some embodiments, from about 30° C. to about 150° C., in some embodiments from about 40° C. to about 100° C., and in some embodiments, in some embodiments from about 100° C. to about 120° C., to form a polymer composition. Any of a variety of melt blending techniques may generally be employed. For example, the components may be supplied separately or in combination to an extruder that includes at least one screw rotatably mounted and received within a barrel (e.g., cylindrical barrel). The extruder may be a single screw or twin screw extruder. For example, one embodiment of a single screw extruder may contain a housing or barrel and a screw rotatably driven on one end by a suitable drive (typically including a motor and gearbox). If desired, a twin-screw extruder may be employed that contains two separate screws. The configuration of the screw is not particularly critical and it may contain any number and/or orientation of threads and channels as is known in the art. For example, the screw typically contains a thread that forms a generally helical channel radially extending around a core of the screw. A feed section and melt section may be defined along the length of the screw. The feed section is the input portion of the barrel where the olefin copolymer(s) and/or therapeutic agent(s) are added. The melt section is the phase change section in which the copolymer is changed from a solid to a liquid-like state. While there is no precisely defined delineation of these sections when the extruder is manufactured, it is well within the ordinary skill of those in this art to reliably identify the feed section and the melt section in which phase change from solid to liquid is occurring. Although not necessarily required, the extruder may also have a mixing section that is located adjacent to the output end of the barrel and downstream from the melting section. If desired, one or more distributive and/or dispersive mixing elements may be employed within the mixing and/or melting sections of the extruder. Suitable distributive mixers for single screw extruders may include, for instance, Saxon, Dulmage, Cavity Transfer mixers, etc. Likewise, suitable dispersive mixers may include Blister ring, Leroy/Maddock, CRD mixers, etc. As is well known in the art, the mixing may be further improved by using pins in the barrel that create a folding and reorientation of the polymer melt, such as those used in Buss Kneader extruders, Cavity Transfer mixers, and Vortex Intermeshing Pin mixers.

If desired, the ratio of the length (“L”) to diameter (“D”) of the screw may be selected to achieve an optimum balance between throughput and blending of the components. The L/D value may, for instance, range from about 10 to about 50, in some embodiments from about 15 to about 45, and in some embodiments from about 20 to about 40. The length of the screw may, for instance, range from about 0.1 to about 5 meters, in some embodiments from about 0.4 to about 4 meters, and in some embodiments, from about 0.5 to about 2 meters. The diameter of the screw may likewise be from about 5 to about 150 millimeters, in some embodiments from about 10 to about 120 millimeters, and in some embodiments, from about 20 to about 80 millimeters. In addition to the length and diameter, other aspects of the extruder may also be selected to help achieve the desired degree of blending. For example, the speed of the screw may be selected to achieve the desired residence time, shear rate, melt processing temperature, etc. For example, the screw speed may range from about 10 to about 800 revolutions per minute (“rpm”), in some embodiments from about 20 to about 500 rpm, and in some embodiments, from about 30 to about 400 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

Once melt blended together, the resulting polymer composition may be in the form of pellets, sheets, fibers, filaments, etc., which may be shaped into the core using a variety of known shaping techniques, such as injection molding, compression molding, nanomolding, overmolding, blow molding, three-dimensional printing, etc. Injection molding may, for example, occur in two main phases—i.e., an injection phase and holding phase. During the injection phase, a mold cavity is filled with the molten polymer composition. The holding phase is initiated after completion of the injection phase in which the holding pressure is controlled to pack additional material into the cavity and compensate for volumetric shrinkage that occurs during cooling. After the shot has built, it can then be cooled. Once cooling is complete, the molding cycle is completed when the mold opens and the part is ejected, such as with the assistance of ejector pins within the mold. Any suitable injection molding equipment may generally be employed in the present disclosure. In one embodiment, an injection molding apparatus may be employed that includes a first mold base and a second mold base, which together define a mold cavity having the shape of the core. The molding apparatus includes a resin flow path that extends from an outer exterior surface of the first mold half through a sprue to a mold cavity. The polymer composition may be supplied to the resin flow path using a variety of techniques. For example, the composition may be supplied (e.g., in the form of pellets) to a feed hopper attached to an extruder barrel that contains a rotating screw (not shown). As the screw rotates, the pellets are moved forward and undergo pressure and friction, which generates heat to melt the pellets. A cooling mechanism may also be provided to solidify the resin into the desired shape of the core (e.g., disc, rod, etc.) within the mold cavity. For instance, the mold bases may include one or more cooling lines through which a cooling medium flows to impart the desired mold temperature to the surface of the mold bases for solidifying the molten material. The mold temperature (e.g., temperature of a surface of the mold) may range from about 30° C. to about 120° C., in some embodiments from about 60° C. to about 110° C., and in some embodiments, from about 30° C. to about 60° C.

As indicated above, another suitable technique for forming a core of the desired shape and size is three-dimensional printing. During this process, the polymer composition may be incorporated into a printer cartridge that is readily adapted for use with a printer system. The printer cartridge may, for example, contains a spool or other similar device that carries the polymer composition. When supplied in the form of filaments, for example, the spool may have a generally cylindrical rim about which the filaments are wound. The spool may likewise define a bore or spindle that allows it to be readily mounted to the printer during use. Any of a variety of three-dimensional printer systems can be employed in the present disclosure. Particularly suitable printer systems are extrusion-based systems, which are often referred to as “fused deposition modeling” systems. For example, the polymer composition may be supplied to a build chamber of a print head that contains a platen and gantry. The platen may move along a vertical z-axis based on signals provided from a computer-operated controller. The gantry is a guide rail system that may be configured to move the print head in a horizontal x-y plane within the build chamber based on signals provided from controller. The print head is supported by the gantry and is configured for printing the build structure on the platen in a layer-by-layer manner, based on signals provided from the controller. For example, the print head may be a dual-tip extrusion head.

Compression molding (e.g., vacuum compression molding) may also be employed. In such a method, a layer of the device may be formed by heating and compressing the polymer compression into the desired shape while under vacuum. More particularly, the process may include forming the polymer composition into a precursor that fits within a chamber of a compression mold, heating the precursor, and compression molding the precursor into the desired layer while the precursor is heated. The polymer composition may be formed into a precursor through various techniques, such as by dry power mixing, extrusion, etc. The temperature during compression may range from about 50° C. to about 120° C., in some embodiments from about 60° C. to about 110° C., and in some embodiments, from about 70° C. to about 90° C. A vacuum source may also apply a negative pressure to the precursor during molding to help ensure that it retains a precise shape. Examples of such compression molding techniques are described, for instance, in U.S. Pat. No. 10,625,444 to Treffer, et al., which is incorporated herein in its entirety by reference thereto.

### II. Therapeutic Agents

### A. Aromatase Inhibitors

Therapeutic agents can also include one or more aromatase inhibitors. Aromatase inhibitors refer to a class of agents that are capable of stopping the production of estrogen in post-menopausal women. Aromatase inhibitors work by blocking the enzyme aromatase, which functions to inhibit the conversion of testosterone and/or androgen into estradiol in the body. Accordingly, the reduction in the action of aromatase reduces the amount of estrogen in the body, therefore less estrogen is available to stimulate the growth of hormone-receptor-positive breast cancer cells. Aromatase inhibitors do not stop the ovaries from making estrogen, therefore, they are more commonly used to treat postmenopausal women. Aromatase inhibitors are known to cause heart problems and bone loss (e.g., osteoporosis).

Suitable examples of aromatase inhibitors include: exemestane, atamestane, formestane, fadrozole, letrozole, pentrozole, anastrozole, vorozole, or combinations thereof. In another embodiment, the aromatase inhibitor can include non-selective aromatase inhibitors such as Aminoglutethimide and Testolactone (Teslac). In yet another embodiment, aromatase inhibitors may include any other selective or non-selective chemical known to people skilled in the art that inhibits the enzyme aromatase and may prevent estrogen from being formed from its metabolic precursors. Aromatase inhibitor polymorphs, isomers, hydrates, solvates, or derivatives thereof are all meant to be encompassed in the scope of the present disclosure and shall be understood to fall under the term “aromatase inhibitor”.

### B. Bisphosphonates

As indicated above, therapeutic agents in the intravaginal ring device can include one or more bisphosphonates dispersed within the core and/or membrane layer(s). Bisphosphonates generally refer to a class of therapeutic agents that slow down or prevent bone loss. Specifically, bisphosphonates inhibit osteoclasts, which are responsible for breaking down and reabsorbing minerals such as calcium from bone via a process known as bone resorption. Bisphosphonates generally allow osteoblasts to work more effectively, thereby improving bone mass. Bisphosphonates are used in the treatment of osteoporosis, Paget's disease of bone, and may also be used to lower calcium levels in cancer patients.

The bisphosphonate class of drugs is based on the phosphate-oxygen-phosphate bond (P—O—P) of pyrophosphate (a widely distributed, natural human metabolite that has a strong affinity for bone). Structurally, bisphosphonates are chemically stable derivatives of inorganic pyrophosphate (PPi), a naturally occurring compound in which two phosphate groups are linked by esterification. Replacing the oxygen with a carbon atom (P—C—P) produces a group of bone-selective drugs that cannot be metabolized by the normal enzymes that break down pyrophosphates. The core structure of bisphosphonates differs only slightly from PPi in that bisphosphonates contain a central nonhydrolyzable carbon; the phosphate groups flanking this central carbon are maintained. Nearly all bisphosphonates in current clinical use also have a hydroxyl group attached to the central carbon (termed the R1 position). The flanking phosphate groups provide bisphosphonates with a strong affinity for hydroxyapatite crystals in bone (and are also seen in PPi), whereas the hydroxyl motif further increases a bisphosphonate's ability to bind calcium. Collectively, the phosphate and hydroxyl groups create a tertiary rather than a binary interaction between the bisphosphonate and the bone matrix, giving bisphosphonates their specificity for bone.

Exemplary bisphosphonates include, but are not limited to, zoledronic acid, risedronate, alendronate, ibandronate, cimadronate, clodronate, tiludronate, minodronate, etidronate, ibandronate, piridronate, pamidronate, 1-fluoro-2-(imidazo-[1,2-α]pyridine-3-yl)-ethyl-bisphosphonic acid, and functional analogues thereof. Bisphosphonate compounds can include first-, second-, and third-generation bisphosphonates. For example, early non-nitrogen containing bisphosphonates, including, etidronate, clodronate, and tiludronate, are considered first-generation bisphosphonates. Second- and third-generation bisphosphonates include alendronate, risedronate, ibandronate, pamidronate, and zoledronate (i.e., zoledronic acid). Such second- and third-generation bisphosphonates have nitrogen containing R2 side chains. The mechanism by which nitrogen-containing bisphosphonates promote osteoclast apoptosis is distinct from that of the non-nitrogen-containing bisphosphonates. For example, nitrogen-containing bisphosphonates bind to and inhibit the activity of farnesyl pyrophosphate synthase, a key regulatory enzyme in the mevalonic acid pathway critical to the production of cholesterol, other sterols, and isoprenoid lipids. As such, the posttranslational modification (isoprenylation) of proteins (including the small guanosine triphosphate—binding proteins Rab, Rac, and Rho, which play central roles in the regulation of core osteoclast cellular activities including stress fiber assembly, membrane ruffling, and survival) is inhibited, ultimately leading to osteoclast apoptosis.

Salts, esters and/or isomers of bisphosphonates are all meant to be encompassed in the scope of the present disclosure and shall be understood to fall under the term “bisphosphonate”.

### C. Corticosteroids

Therapeutic agents can also include one or more corticosteroids, including glucocorticoids. Glucocorticoids are defined as a subgroup of corticosteroids. Glucocorticoids, sometimes also named glucocorticosteroids, are a class of steroid hormones that bind to the glucocorticoid receptor and are part of the feedback mechanism of the immune system that turns down immune activity, (e.g., inflammation). In medicine they are used to treat diseases that are caused by an overactive immune system, such as allergies, asthma, autoimmune diseases, and sepsis. They also interfere with some of the abnormal mechanisms in cancer cells, so that they are also used to treat cancer. Upon binding the glucocorticoid receptor, the activated glucocorticoid receptor complex up-regulates the expression of anti-inflammatory proteins in the nucleus by a process known as transactivation and represses the expression of pro-inflammatory proteins in the cytosol by attenuating actions on gene induction (via NF-κB, AP1, jun-jun-homoclimers etc.).

Suitable examples of glucocorticoids include hydrocortisone, cortisone acetate, cortisone/cortisol, fluorocortolon, prednisone, prednisolone, methylprednisolone, triamcinolone, dexamethasone, betamethasone, paramethasone. Glucocorticoid polymorphs, isomers, hydrates, solvates, or derivatives thereof are all meant to be encompassed in the scope of the present disclosure and shall be understood to fall under the term “glucocorticoid”.

### D. Selective Estrogen Receptor Modulators (SERMs)

Therapeutic agents can also include SERMs. SERMs are agents that bind to estrogen receptors but that have the ability to act either as agonists or antagonists in different tissues. For example, in certain SERMs act as agonists on the bone and uterus estrogen receptors and act as antagonists on the breast estrogen receptors. Growth of certain forms of cancers (e.g., breast cancers) may be dependent on estrogen. Accordingly, selective SERMS that act as antagonists on breast tissue are used in the treatment of breast cancer. Additionally, SERMs can be useful in preventing post-menopausal osteoporosis and certain metastatic breast cancers. SERMs are small ligands of the estrogen receptor that are capable of inducing a wide variety of conformational changes in the receptor and thereby eliciting a variety of distinct biological profiles. SERMs not only affect the growth of breast cancer tissue but also influence other physiological processes.

SERMs modulate the proliferation of uterine tissue, skeletal bone density, and cardiovascular health, including plasma cholesterol levels. In general, estrogen stimulates breast and endometrial tissue proliferation, enhances bone density, and lowers plasma cholesterol. Many SERMs are bifunctional in that they antagonize some of these functions while stimulating others. For example, tamoxifen, which is a partial agonist/antagonist at the estrogen receptor inhibits estrogen-induced breast cancer cell proliferation but stimulates endometrial tissue growth and prevents bone loss.

Suitable SERMs include ospemifene, raloxifene, tamoxifene, toremifene, lasofoxifene, bazedoxifene, clomiphene citrate, ormeloxifenem, tibolone, idoxifene, or combinations thereof. SERM polymorphs, isomers, hydrates, solvates, or derivatives thereof are all meant to be encompassed in the scope of the present disclosure and shall be understood to fall under the term “SERM”. Raloxifene and tamoxifene are some of the most commonly prescribed and utilized SERMs.

Raloxifene is an estrogen agonist/antagonist, which belongs to the benzothiophene class of compounds. Raloxifene is represented by structural formula (1).

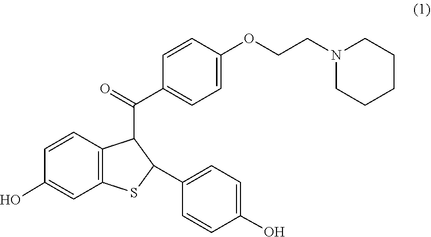 [SMILES: O=C(c1ccc(OCCN2CCCCC2)cc1)C1c2ccc(O)cc2SC1c1ccc(O)cc1]

A chemical name for raloxifene hydrochloride is methanone, [6-hydroxy-2-(4-hydroxyphenyl)benzo[b]thiene-3-yl]-[4-[2-(1-piperidinyl)ethoxy]phenyl]-, hydrochloride. Raloxifene hydrochloride has the empirical formula C₂₈H₂₇NO₄S·HCl, corresponding to a molecular weight of 510.05. Raloxifene hydrochloride is an off-white to pale yellow solid that is very slightly soluble in water, the water solubility being approximately 0.3 g/ml at 25° C., and significantly lower in simulated gastric fluid (SGF) USP (0.003 mg/ml) and simulated intestinal fluid (SIF) USP (0.002 mg/ml), at 37° C. Raloxifene and its derivatives as anti-estrogenic or anti-androgenic compounds are disclosed in U.S. Pat. No. 4,418,068.

Tamoxifen is the trans-isomer of a triphenylethylene derivative. The chemical name is (Z)2-[4-(1,2-diphenyl-1-butenyl)phenoxy]-N,N-dimethylethanamine 2-hydroxy-1,2,3-propanetricarboxylate (1:1). The structural formula, empirical formula, and molecular weight are as follows:

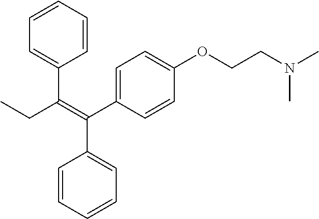 [SMILES: CC/C(=C(\c1ccccc1)c1ccc(OCCN(C)C)cc1)c1ccccc1]

The empirical formula of tamoxifene is C₃₂H₃₇NO₈ and it has a molecular weight of 563.62 Tamoxifen citrate has a pKa′ of 8.85. The equilibrium solubility in water at 37° C. is 0.5 mg/mL, and is 0.2 mg/mL in 0.02 N HCl at 37° C.

### E. Other Therapeutic Agents

Therapeutic agents utilized in the intravaginal device can further include any therapeutic agent suitable for treating cancers, especially hormone-dependent cancers. For example, the therapeutic agent can include certain hormones administered for cancer treatments. Additional therapeutic agents can include those used to treat or offset side effects from cancer treatment medications.

### III. Membrane Layer(s)

As indicated above, the intravaginal ring device can optionally include one or more membrane layers (e.g., a first membrane layer) that is positioned adjacent to an outer surface of a core. Additional membrane layers (e.g., a second membrane layer, a third membrane layer, etc.) may be layered on the core as desired. The number of membrane layers may vary depending on the particular configuration of the device, the nature of the therapeutic agent, and the desired release profile.

Regardless of the particular configuration employed, the membrane polymer matrix contains at least one ethylene vinyl acetate copolymer, such as described in more detail above. The vinyl acetate content of the copolymer may be selectively controlled to be within a range of from about 10 wt. % to about 60 wt. %, in some embodiments from about 20 wt. % to about 60 wt. %, in some embodiments from about 25 wt. % to about 50 wt. %, in some embodiments from about 30 wt. % to about 48 wt. %, and in some embodiments, from about 35 wt. % to about 45 wt. % of the copolymer. Conversely, the ethylene content of the copolymer may likewise be within a range of from about 40 wt. % to about 90 wt. %, in some embodiments from about 40 wt. % to about 80 wt. %, in some embodiments from about 50 wt. % to about 75 wt. %, in some embodiments from about 50 wt. % to about 80 wt. %, in some embodiments from about 52 wt. % to about 70 wt. %, and in some embodiments, from about 55 wt. % to about 65 wt. %. The melt flow index of the ethylene vinyl acetate copolymer(s) and resulting polymer matrix may also range from about 0.2 to about 400 g/10 min, in some embodiments 0.2 to about 100 g/10 min, in some embodiments from about 5 to about 90 g/10 min, in some embodiments from about 10 to about 80 g/10 min, and in some embodiments, from about 30 to about 70 g/10 min, as determined in accordance with ASTM D1238-20 at a temperature of 190° C. and a load of 2.16 kilograms. The melting temperature of the ethylene vinyl acetate copolymer may also range from about 40° C. to about 140° C., in some embodiments from about 50° C. to about 125° C., and in some embodiments, from about 60° C. to about 120° C., as determined in accordance with ASTM D3418-15. The density of the ethylene vinyl acetate copolymer(s) may also range from about 0.900 to about 1.00 gram per cubic centimeter (g/cm³), in some embodiments from about 0.910 to about 0.980 g/cm³, and in some embodiments, from about 0.940 to about 0.970 g/cm³, as determined in accordance with ASTM D1505-18. Particularly suitable examples of ethylene vinyl acetate copolymers that may be employed include those available from Celanese under the designation ATEVA® (e.g., ATEVA® 4030AC); Dow under the designation ELVAX® (e.g., ELVAX® 40W); and Arkema under the designation EVATANE® (e.g., EVATANE 40-55). In embodiments, the ethylene vinyl acetate copolymer in the membrane polymer matrix is from about 20 wt. % to about 90 wt. %, such as from about 30 wt. % to about 80 wt. %, such as from about 40 wt. % to about 70 wt. %.

In certain cases, ethylene vinyl acetate copolymer(s) constitute the entire polymer content of the membrane polymer matrix. In other cases, however, it may be desired to include other polymers, such as other hydrophobic polymers. When employed, it is generally desired that such other polymers constitute from about 0.001 wt. % to about 30 wt. %, in some embodiments from about 0.01 wt. % to about 20 wt. %, and in some embodiments, from about 0.1 wt. % to about 10 wt. % of the polymer content of the polymer matrix. In such cases, ethylene vinyl acetate copolymer(s) may constitute about from about 70 wt. % to about 99.999 wt. %, in some embodiments from about 80 wt. % to about 99.99 wt. %, and in some embodiments, from about 90 wt. % to about 99.9 wt. % of the polymer content of the polymer matrix. The membrane polymer matrix typically constitutes from about 50 wt. % to 99 wt. %, in some embodiments, from about 55 wt. % to about 98 wt. %, in some embodiments from about 60 wt. % to about 96 wt. %, and in some embodiments, from about 70 wt. % to about 95 wt. % of a membrane layer.

To help further control the release rate from the implantable medical device, a hydrophilic compound may also be incorporated into the membrane layer(s) that is soluble and/or swellable in water. When employed, the weight ratio of the ethylene vinyl acetate copolymer(s) the hydrophilic compounds within the membrane layer may range about 0.25 to about 200, in some embodiments from about 0.4 to about 80, in some embodiments from about 0.8 to about 20, in some embodiments from about 1 to about 16, and in some embodiments, from about 1.2 to about 10. Such hydrophilic compounds may, for example, constitute from about 1 wt. % to about 60 wt. %, in some embodiments from about 2 wt. % to about 50 wt. %, and in some embodiments, from about 5 wt. % to about 40 wt. % of the core, while ethylene vinyl acetate copolymer(s) typically constitute from about 40 wt. % to about 99 wt. %, in some embodiments from about 50 wt. % to about 98 wt. %, and in some embodiments, from about 60 wt. % to about 95 wt. % of the core. Suitable hydrophilic compounds may include, for instance, polymers, non-polymeric materials (e.g., glycerin, saccharides, sugar alcohols, salts, etc.), etc. Examples of suitable hydrophilic polymers include, for instance, sodium, potassium and calcium alginates, carboxymethylcellulose, agar, gelatin, polyvinyl alcohols, polyalkylene glycols (e.g., polyethylene glycol), collagen, pectin, chitin, chitosan, poly-1-caprolactone, polyvinylpyrrolidone, poly(vinylpyrrolidone-co-vinyl acetate), polysaccharides, hydrophilic polyurethane, polyhydroxyacrylate, dextran, xanthan, hydroxypropyl cellulose, methylcellulose, proteins, ethylene vinyl alcohol copolymers, water-soluble polysilanes and silicones, water-soluble polyurethanes, etc., as well as combinations thereof. Particularly suitable hydrophilic polymers are polyalkylene glycols, such as those having a molecular weight of from about 100 to 500,000 grams per mole, in some embodiments from about 500 to 200,000 grams per mole, and in some embodiments, from about 1,000 to about 100,000 grams per mole. Specific examples of such polyalkylene glycols include, for instance, polyethylene glycols, polypropylene glycols polytetramethylene glycols, polyepichlorohydrins, etc.

Optionally, the membrane layer(s) can include a plurality of water-soluble particles distributed within a membrane polymer matrix. The particle size of the water-soluble particles is controlled to help achieve the desired delivery rate. More particularly, the median diameter (D50) of the particles is about 100 micrometers or less, in some embodiments about 80 micrometers or less, in some embodiments about 60 micrometers or less, and in some embodiments, from about 1 to about 40 micrometers, such as determined using a laser scattering particle size distribution analyzer (e.g., LA-960 from Horiba). The particles may also have a narrow size distribution such that 90% or more of the particles by volume (D90) have a diameter within the ranges noted above. In addition to controlling the particle size, the materials employed to form the water-soluble particles are also selected to achieve the desired release profile. More particularly, the water-soluble particles generally contain a hydroxy-functional compound that is not polymeric. The term “hydroxy-functional” generally means that the compound contains at least one hydroxyl group, and in certain cases, multiple hydroxyl groups, such as 2 or more, in some embodiments 3 or more, in some embodiments 4 to 20, and in some embodiments, from 5 to 16 hydroxyl groups. The term “non-polymeric” likewise generally means that the compound does not contain a significant number of repeating units, such as no more than 10 repeating units, in some embodiments no or more than 5 repeating units, in some embodiments no more than 3 repeating units, and in some embodiments, no more than 2 repeating units. In some cases, such a compound lacks any repeating units. Such non-polymeric compounds thus a relatively low molecular weight, such as from about 1 to about 650 grams per mole, in some embodiments from about 5 to about 600 grams per mole, in some embodiments from about 10 to about 550 grams per mole, in some embodiments from about 50 to about 500 grams per mole, in some embodiments from about 80 to about 450 grams per mole, and in some embodiments, from about 100 to about 400 grams per mole. Particularly suitable non-polymeric, hydroxy-functional compounds that may be employed in the present disclosure include, for instance, saccharides and derivatives thereof, such as monosaccharides (e.g., dextrose, fructose, galactose, ribose, deoxyribose, etc.); disaccharides (e.g., sucrose, lactose, maltose, etc.); sugar alcohols (e.g., xylitol, sorbitol, mannitol, maltitol, erythritol, galactitol, isomalt, inositol, lactitol, etc.); and so forth, as well as combinations thereof. If utilized, the water-soluble particles typically constitute from about 1 wt. % to about 50 wt. %, in some embodiments from about 2 wt. % to about 45 wt. %, in some embodiments from about 4 wt. % to about 40 wt. %, and in some embodiments, from about 5 wt. % to about 30 wt. % of a membrane layer.

When employing multiple membrane layers, it is typically desired that each membrane layer contains a polymer matrix includes an ethylene vinyl acetate copolymer. Additionally, each of the membrane layers can include a plurality of water-soluble particles distributed within a membrane polymer matrix that includes an ethylene vinyl acetate copolymer. For example, a first membrane layer may contain first water-soluble particles distributed within a first membrane polymer matrix and a second membrane layer may contain second water-soluble particles distributed within a second membrane polymer matrix. In such embodiments, the first and second polymer matrices may each contain an ethylene vinyl acetate copolymer. The water-soluble particles and ethylene vinyl acetate copolymer(s) within one membrane layer may be the same or different than those employed in another membrane layer. In one embodiment, for instance, both the first and second membrane polymer matrices employ the same ethylene vinyl acetate copolymer(s) and the water-soluble particles within each layer have the same particle size and/or are formed from the same material. Likewise, the ethylene vinyl acetate copolymer(s) used in the membrane layer(s) may also be the same or different the hydrophobic polymer(s) employed in the core. In one embodiment, for instance, both the core and the membrane layer(s) employ the same ethylene vinyl acetate copolymer. In yet other embodiments, the membrane layer(s) may employ an ethylene vinyl acetate copolymer that has a lower melt flow index than a hydrophobic polymer employed in the core. Among other things, this can further help control the release of the therapeutic agent from the device. For example, the ratio of the melt flow index of a hydrophobic polymer employed in the core to the melt flow index of an ethylene vinyl acetate copolymer employed in the membrane layer(s) may be from about 1 to about 20, in some embodiments about 2 to about 15, and in some embodiments, from about 4 to about 12.

If desired, membrane layer(s) used in the device may optionally contain a therapeutic agent, such as described above, which is also dispersed within the membrane polymer matrix. The therapeutic agent in the membrane layer(s) may be the same or different than the therapeutic agent employed in the core. When such a therapeutic agent is employed in a membrane layer, the membrane layer generally contains the therapeutic agent in an amount such that the ratio of the concentration (wt. %) of the therapeutic agent in the core to the concentration (wt. %) of the therapeutic agent in the membrane layer is greater than 1, in some embodiments about 1.5 or more, and in some embodiments, from about 1.8 to about 4. When employed, therapeutic agents typically constitute only from about 1 wt. % to about 40 wt. %, in some embodiments from about 5 wt. % to about 35 wt. %, and in some embodiments, from about 10 wt. % to about 30 wt. % of a membrane layer. Of course, in other embodiments, the membrane layer is generally free of therapeutic agents prior to release from the core. When multiple membrane layers are employed, each membrane layer may generally contain the therapeutic agent in an amount such that the ratio of the weight percentage of the therapeutic agent in the core to the weight percentage of the therapeutic agent in the membrane layer is greater than 1, in some embodiments about 1.5 or more, and in some embodiments, from about 1.8 to about 4.

The membrane layer(s) may also optionally contain one or more excipients as described above, such as radiocontrast agents, bulking agents, plasticizers, surfactants, crosslinking agents, flow aids, colorizing agents (e.g., chlorophyll, methylene blue, etc.), antioxidants, stabilizers, lubricants, other types of antimicrobial agents, preservatives, etc. to enhance properties and processability. When employed, the optional excipient(s) typically constitute from about 0.01 wt. % to about 60 wt. %, and in some embodiments, from about 0.05 wt. % to about 50 wt. %, and in some embodiments, from about 0.1 wt. % to about 40 wt. % of a membrane layer.

The membrane layer(s) may be formed using the same or a different technique than used to form the core, such as by hot-melt extrusion, compression molding (e.g., vacuum compression molding), injection molding, solvent casting, dip coating, spray coating, microextrusion, coacervation, etc. In one embodiment, a hot-melt extrusion technique may be employed. The core and membrane layer(s) may also be formed separately or simultaneously. In one embodiment, for instance, the core and membrane layer(s) are separately formed and then combined together using a known bonding technique, such as by stamping, hot sealing, adhesive bonding, etc. Compression molding (e.g., vacuum compression molding) may also be employed to form the intravaginal ring device. As described above, the core and membrane layer(s) may be each individually formed by heating and compressing the respective polymer compression into the desired shape while under vacuum. Once formed, the core and membrane layer(s) may be stacked together to form a multi-layer precursor and thereafter and compression molded in the manner as described above to form the resulting intravaginal ring device.

### IV. Use of Device

The intravaginal ring device of the present disclosure may be used in a variety of different ways to treat prohibit and/or treat a condition, disease, or cosmetic state in a patient. The term “intravaginal ring device” as used herein, is intended to cover a variety of implantable or insertable devices and associated methods of use. For example, the intravaginal ring device can be inserted intravaginally using standard techniques. In such embodiments, the delivery route may be intravaginal. As noted above, the intravaginal ring device may be particularly suitable for delivering an aromatase inhibitor for treatment of hormone receptor-positive cancers. The device may also be employed together with current systemic therapies for menopausal and post-menopausal women, including hormone replacement therapies, cancer treatments, (e.g., those for treatment of post-menopausal cancers, such as breast cancer). The device can also be employed together with other therapies for cancer treatments including chemotherapy, external radiation, and/or surgery.

Furthermore, the intravaginal ring device can be used to provide one or more aromatase inhibitors while the patient is undergoing cancer therapy or hormone therapy (e.g., the administration of estrogen or estrogen analogues or other hormones). In such embodiments, these additional therapeutic agents can be administered to the patient in a variety of dosage forms, including, oral dosage forms, intravenous dosage forms, subcutaneous dosage forms, including depot injections, hydrogel injections, intramuscular injections, etc., or intravaginal dosage forms. Additional therapeutic agents can be administered via any suitable route and can be used in combination with the intravaginal ring device disclosed herein.

The intravaginal ring device can be in different forms, and encompasses such devices including an intrauterine system (IUS) (e.g., intrauterine device), a helical coil, a spring, a rod, a cylinder, and/or a vaginal ring. The intravaginal ring device can include a core and any membrane layers as disclosed herein. For example, a method of manufacture of the ring-shaped device includes extrusion of the core containing the aromatase inhibitor or co-extrusion of the core containing aromatase inhibitor and one or more membrane layers, to render a rod or fiber. The rod/fiber can then be cut into pieces of required lengths and assembled into a ring-shaped device via any suitable molding procedure. For example, an intravaginal ring device in the form of a rod can be formed and the ends of the rod can be joined together to form a ring. Additional membrane layers, as required, can be incorporated and/or layered on the vaginal ring.

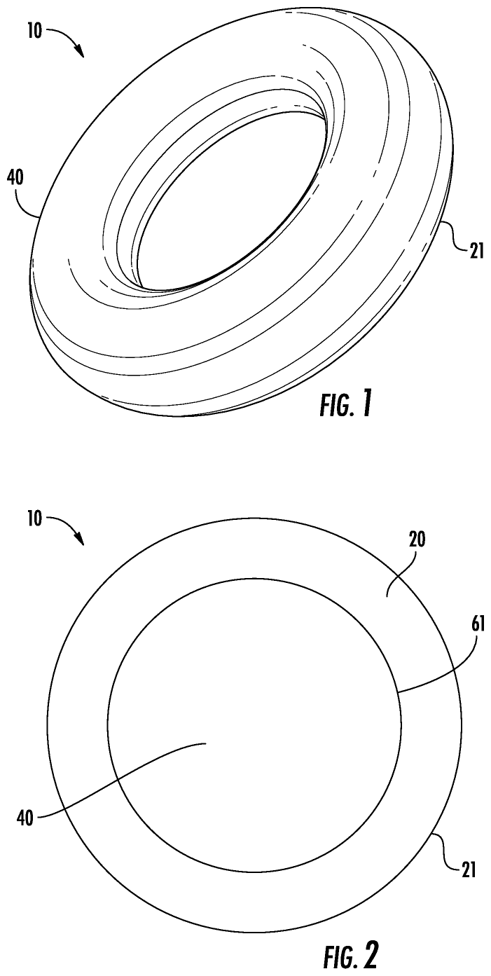

Referring to FIGS. 1-2, for example, an intravaginal ring device 10 is shown that contains a core 40 having a generally circular cross-sectional shape. The core 40 defines an outer surface 61 about which a membrane layer 20 is circumferentially disposed. Similar to the core 40, the membrane layer 20 also has a generally circular cross-section shape and is configured so that it covers the entire outer surface of the core 40. During use of the device 10, a therapeutic agent is capable of being released from the core 40 and through the membrane layer 20 so that it exits from an external surface 21 of the device 10.

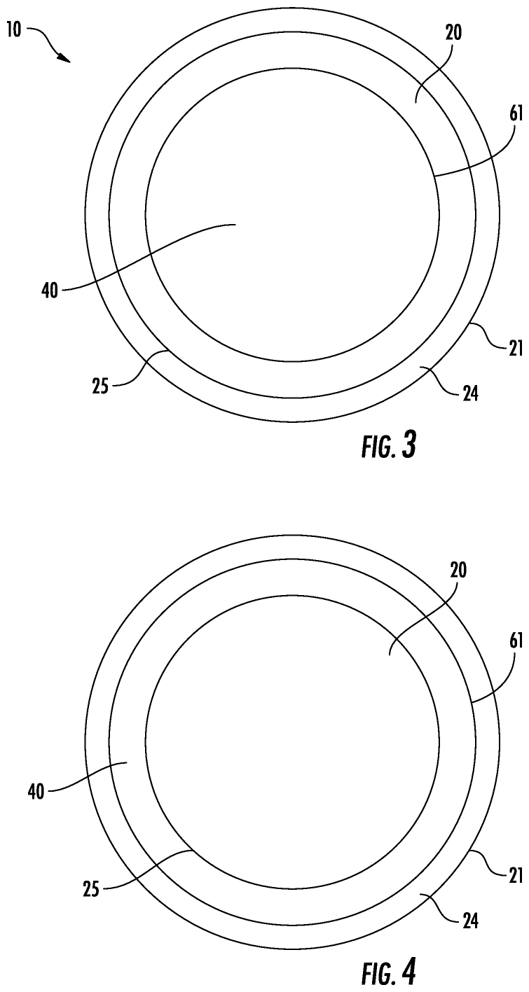

Of course, in other embodiments, the device 10 may contain multiple membrane layers. In the device 10 of FIGS. 1-2, for example, one or more additional membrane layers (not shown) may be disposed over the membrane layer 20 to help further control release of the therapeutic agent. For example, as shown in FIG. 3, a second membrane layer 24 is positioned adjacent to an outer surface 25 of the first membrane layer 20. In other embodiments, specifically as shown in FIG. 4, the device 10 may be configured so that the core 40 is positioned or sandwiched between separate membrane layers 20, 24. As shown, the core 40 is positioned on an outer surface 25 of the first membrane layer 20. A second membrane layer 24 is disposed on the outer surface 61 of the core 40. Furthermore, if desired, the membrane layers may be disposed on the core such that they do not cover the entire exposed area of the core. Further, membrane layers can be disposed around a certain portion of the core, while a different membrane layer can be disposed around another portion of the core.

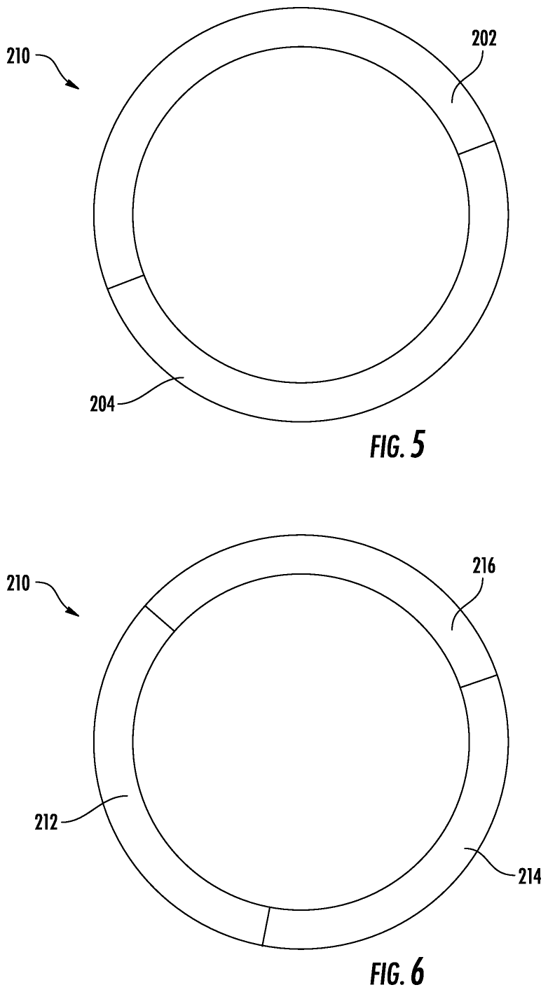

In certain embodiments, a multi-compartment ring can be formed. An example intravaginal ring device 200 is shown in FIG. 5 having at least two compartments 202, 204, while the intravaginal ring device 210 as shown in FIG. 6 includes at least three compartments 212, 214, 216. While two and three compartment examples are shown, the disclosure is not so limited. Indeed, the intravaginal ring devices can include a plurality of compartments. In fact, any number of compartments or sections can be joined together to form an intravaginal ring device as provided herein. Furthermore, any suitable materials can be used or placed between compartments when molding the ring. Each compartment of the intravaginal ring device (e.g., 202, 204 or 212, 214, 216) can be the same or different. For example, for delivery of a combination of therapeutic agents (e.g., one or more aromatase inhibitors with an additional therapeutic agent), the compartments can contain different types or amounts of therapeutic agents. One or more compartments can contain aromatase inhibitor, while one or more other compartments of the ring are formulated with additional therapeutic agents (e.g., SERMs, glucocorticosteroids, and/or bisphosphonates). Additionally, one or more hormones can be incorporated into the compartments of the intravaginal ring device as disclosed herein. In such embodiments, the intravaginal ring device can provide combination therapy for patients.

In certain other embodiments, multi-compartment intravaginal ring devices can be formed having different types and/or amounts of aromatase inhibitor dispersed in each compartment. Such embodiments provide for the delivery of multiple aromatase inhibitor compounds from the intravaginal ring device. In certain embodiments, the amount of aromatase inhibitor delivered from each compartment can vary. Indeed, each compartment may be formulated with a different core polymer matrix and/or membrane layer in order to affect the release rate of aromatase inhibitor from each compartment. For example, certain compartments can be configured to release aromatase inhibitor faster in order to reach an initial steady state concentration, while the remaining compartments can be formulated to release aromatase inhibitor more slowly such that sustained delivery of one or more aromatase inhibitors over a period of time can be achieved.

The compartments disclosed herein can include one or more membrane layers as disclosed herein. The membrane layers of the compartments can be varied in order to further effect release of the dispersed therapeutic agents from the compartments.

The size of the intravaginal ring device can vary. For example, the cross-sectional diameter of the vaginal ring will typically range from about 1.5 mm to about 6 mm, such as from about 2 mm to about 5 mm, such as about 4 mm. The ring diameter of the vaginal ring will typically range from about 2.5 cm to about 7.5 cm, such as from about 3 cm to about 6 cm, such as about 5 cm. Given that the therapeutic agent is typically administered from an outer surface of the ring, the vaginal rings disclosed herein can be sized to have a total surface area ranging from about 10 cm² to about 30 cm², such as about 20 cm².

Through selective control over the particular nature of the device and the manner in which it is formed, the resulting device can be effective for sustained release of one or more aromatase inhibitors over a prolonged period of time. For example, the intravaginal ring device can release the therapeutic agent(s) for a time period of about 5 days or more, in some embodiments about 10 days or more, in some embodiments from about 21 days or more, and in some embodiments, from about 25 days to about 50 days (e.g., about 30 days). In certain embodiments, the device can release the therapeutic agent(s) for a time period for about 3 months or more, such as about 6 months or more, such as about 12 month or more, and in some embodiments, from about 12 months to about 36 months. Further, the therapeutic agent(s) can be released in a controlled manner (e.g., zero order or near zero order) over the course of the release time period. After a time period of 21 days, for example, the cumulative release ratio of the intravaginal ring device may be from about 20% to about 70%, in some embodiments from about 30% to about 65%, and in some embodiments, from about 40% to about 60%. Likewise, after a time period of 30 days, the cumulative release ratio of the intravaginal ring device may still be from about 40% to about 85%, in some embodiments from about 50% to about 80%, and in some embodiments, from about 60% to about 80%. The “cumulative release ratio” may be determined by dividing the amount of the therapeutic agent released at a particulate time interval by the total amount of therapeutic agent initially present, and then multiplying this number by 100.

Of course, the actual dosage level of the aromatase inhibitor delivered will vary depending on the particular aromatase inhibitor employed and the time period for which it is intended to be released. The dosage level is generally high enough to provide a therapeutically effective amount of the aromatase inhibitor to render a desired therapeutic outcome, i.e., a level or amount effective to reduce or alleviate symptoms of the condition for which it is administered. The exact amount necessary will vary, depending on the subject being treated, the age and general condition of the subject to which the aromatase inhibitor is to be delivered, the capacity of the subject's immune system, the degree of effect desired, the severity of the condition being treated, the particular aromatase inhibitor selected and mode of administration of the composition, among other factors. For example, an effective amount will typically range from about 0.1 mg to about 10 mg per day, such as from about 0.5 mg to about 5 mg per day, such as about 1 mg per of the aromatase inhibitor delivered per day. Additional therapeutic agents (e.g., bisphosphonates) can also be loaded into the intravaginal ring and co-administered therefrom with one or more aromatase inhibitors. Effective amounts for such additional therapeutic agents can typically range from about 0.01 mg to about 4 mg per day, such as from about 0.05 mg to about 3 mg per day, such as about 0.1 mg of the additional therapeutic agents delivered per day.

The amount of aromatase inhibitor loaded into the device can vary. For example, vaginal ring implants configured to be inserted vaginally and retained for periods of time ranging from a few days to a few weeks, but generally less than 2 months, the core can be loaded with from about 50 mg to about 1000 mg of one or more aromatase inhibitors. The core may also be loaded with from about 800 mg to about 2500 mg of aromatase inhibitor. Additionally, the amount of aromatase inhibitor loaded into the core can be modified (e.g., increased and/or decreased) depending on the amount of implantation time desired or route of implantation. Additionally, the amount of aromatase inhibitor loaded into the core can be modified based on the use of additional therapeutic agents in addition to the aromatase inhibitors. For example, an increase in the amount of aromatase inhibitor loaded into the core can be increased with the implant includes or is co-administered with one or more therapeutic agents known to increase estrogen production.

If desired, the device may be sealed within a package (e.g., sterile blister package) prior to use. The materials and manner in which the package is sealed may vary as is known in the art. In one embodiment, for instance, the package may contain a substrate that includes any number of layers desired to achieve the desired level of protective properties, such as 1 or more, in some embodiments from 1 to 4 layers, and in some embodiments, from 1 to 3 layers. Typically, the substrate contains a polymer film, such as those formed from a polyolefin (e.g., ethylene copolymers, propylene copolymers, propylene homopolymers, etc.), polyester (e.g., polyethylene terephthalate, polyethylene naphthalate, polybutylene terephthalate, etc.), vinyl chloride polymer, vinyl chloridine polymer, ionomer, etc., as well as combinations thereof. One or multiple panels of the film may be sealed together (e.g., heat sealed), such as at the peripheral edges, to form a cavity within which the device may be stored. For example, a single film may be folded at one or more points and sealed along its periphery to define the cavity within with the device is located. To use the device, the package may be opened, such as by breaking the seal, and the device may then be removed and implanted into a patient.

### EXAMPLES 1-5

Ateva® 1820, 2820A and 4030AC was compounded with Exemestane via 11 mm twin-screw extruder. Three different loading percentages i.e., 10, 40 and 60 were selected for Exemestane as shown in Table 1. A total of five different formulations were produced, and the diameter of the compounded filaments varied from around 1.8 mm to 3.1 mm. For drug elution testing filaments were cut to about 0.3 cm long pieces to perform in vitro release.

TABLE 1

| | Example | Example | Example | Example | Example |
|---|---|---|---|---|---|
| | 1 | 2 | 3 | 4 | 5 |
| Exem- | 10% | 40% | 60% | 10% | 10% |
| estane | | | | | |
| Ateva ® | 90% | 60% | 40% | — | — |
| 4030AC | | | | | |
| Ateva ® | — | — | — | 90% | — |
| 2820A | | | | | |
| Ateva ® | — | — | — | — | 90% |
| 1820 | | | | | |

The release of exemestane from rods into Phosphate buffer 150 mM was measured in a shaking incubator maintained at 37° C. At regular intervals, the buffer was exchanged with fresh buffer, and the removed buffer characterized using High performance liquid chromatography (HPLC) technique.

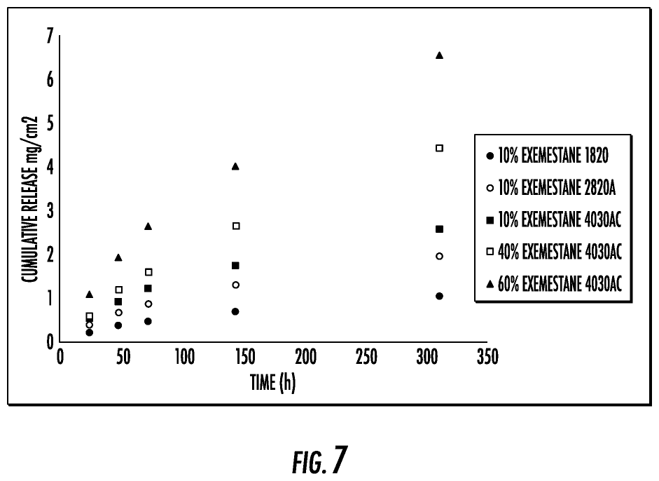

FIG. 7 illustrates the cumulative release for Examples 1-5 as referenced above in Table 1. For reference the following Examples correspond to the legend in FIG. 7 as follows: Example 5 is 10% Exemestane 1820; Example 4 is 10% Exemestane 2820A; Example 1 is 10% Exemestane 4030AC; Example 2 is 40% Exemestane 4030AC; and Example 3 is 60% Exemestane 4030AC.

### EXAMPLES 6-8

### Disc Preparation

Rods from Examples 1, 3, and 4 were reprocessed into discs using a vacuum compression molding (VCM) apparatus (MeltPrep). The thickness of discs prepared was about 1 mm and diameter was about 20 mm. The disc (core) formulations are shown in Table 2.

### Membrane Application

Membranes of Ateva® 2820A were applied on the discs via VCM. The thickness of the discs after membrane application was about 1.55 mm. The diameter remained about 20 mm. The core and membrane formulations are presented in Table 2. The discs were then cut to four quadrants and a membrane of Ateva® 4030AC was then applied to the exposed surface of each quadrant as a sealing edge membrane. The average surface area of surface where the Ateva® 2820A membrane was applied on a quadrant was about 1.9 cm². The average surface area where the Ateva® 4030AC of the sealing edge membrane was applied on a quadrant was about 0.1 cm². The ratio of the Ateva® 2820A membrane surface area as compared to the Ateva® 4030AC sealing edge area was about 19. Given the small amount of Ateva® 4030AC used to seal the edges of the quadrant, the majority of drug diffusion out of the quadrant implant occurs through the Ateva® 2820A. The Ateva® 4030AC sealing edge membrane was only utilized as a sealant to cover the exposed surfaces of a quadrant and substantially no drug diffusion occurs through the Ateva® 4030AC sealing edge membrane.

TABLE 2

| | EVA/Exemestane core (Discs) | | |
|---|---|---|---|
| | Example 6 | Example 7 | Example 8 |
| Exemestane | 10% | 60% | 10% |
| Ateva ® | 90% | 40% | — |
| 4030 | | | |
| Ateva ® | — | — | 90% |
| 2820 | | | |
| | Disc Membrane | | |
| | Ateva ® 2820 | Ateva ® 2820 | Ateva ® 2820 |
| | Sealing Edge Membrane | | |
| | EVA 4030 | EVA 4030 | EVA 4030 |

### EXAMPLES 9-11

### Disc Preparation

EVA/Exemestane discs were prepared by using the extruded filaments of Examples 1, 3, and 4 via vacuum compression molding. The thickness of the discs prepared was about 1 mm and the diameter was about 20 mm. The disc (core) formulations are present in Table 3. Each disc was cut into four (4) quadrants. No membrane layers were applied to Examples 9-11.

TABLE 3

| | EVA/Exemestane core (Discs) | | |
|---|---|---|---|
| | Formulation 1 | Formulation 3 | Formulation 4 |
| Exemestane | 10% | 60% | 10% |
| EVA 4030 | 90% | 40% | — |
| EVA 2820 | — | — | 90% |

### Elution Studies

The release of exemestane from Examples 6-11 into Phosphate buffer saline (PBS) was measured in a shaking incubator maintained at 37° C. At regular intervals, the buffer was exchanged with fresh buffer, and the removed buffer characterized using High performance liquid chromatography (HPLC) technique.

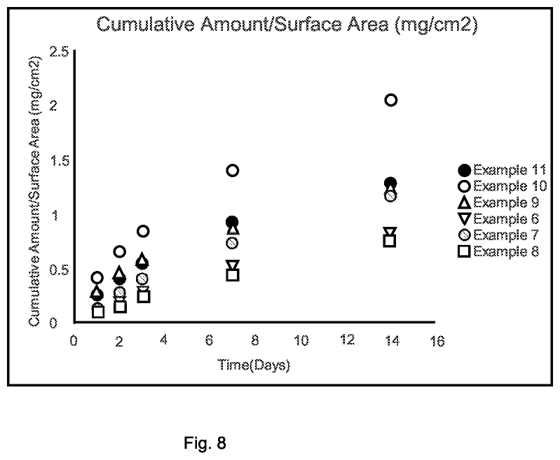

FIG. 8 illustrates the cumulative release for Examples 6-11 as referenced above in Tables 2-3.

These and other modifications and variations of the present disclosure may be practiced by those of ordinary skill in the art, without departing from the spirit and scope of the present disclosure. In addition, it should be understood that aspects of the various embodiments may be interchanged both in whole or in part. Furthermore, those of ordinary skill in the art will appreciate that the foregoing description is by way of example only, and is not intended to limit the disclosure so further described in such appended claims.

What is claimed is:

1. An intravaginal ring device for delivering one or more aromatase inhibitors, the intravaginal ring device comprising:
a core comprising a core polymer matrix within which is dispersed a therapeutic agent comprising one or more aromatase inhibitors, the core polymer matrix containing an ethylene vinyl acetate copolymer, wherein the ethylene vinyl acetate copolymer has a vinyl acetate content of from about 10 wt. % to about 60 wt. % and/or a melting temperature of from about 40° C. to about 120° C. as determined in accordance with ASTM D3418-15.

2. The intravaginal ring device of claim 1, wherein the core polymer matrix has a melt flow index of from about 1 to about 400 grams per 10 minutes as determined in accordance with ASTM D1238-20 at a temperature of 190° C. and a load of 2.16 kilograms.

3. The intravaginal ring device of claim 1, wherein the core polymer matrix further includes one or more hydrophobic polymers.

4. The intravaginal ring device of claim 1, wherein the ethylene vinyl acetate copolymer in the core polymer matrix is from about 20 wt. % to about 90 wt. %.

5. The intravaginal ring device of claim 1, wherein the core polymer matrix includes a first ethylene vinyl acetate copolymer and a second ethylene vinyl acetate copolymer.

6. The intravaginal ring device of claim 1, wherein the one or more aromatase inhibitors comprise anastrozole, exemestane, letrozole, and combinations thereof.

7. The intravaginal ring device of claim 1, further comprising at least one other therapeutic agent.

8. The intravaginal ring device of claim 7, wherein the at least one other therapeutic agent comprises one or more bisphosphonates, one or more glucocorticoids, one or more selective estrogen receptor modulators, and combinations thereof.

9. The intravaginal ring device of claim 1, wherein the intravaginal ring device includes one or more compartments.

10. The intravaginal ring device of claim 1, wherein the ethylene vinyl acetate copolymer has a vinyl acetate content of from about 15 wt. % to about 35 wt. %.

11. The intravaginal ring device of claim 1, wherein the therapeutic agent comprises from about 5 wt. % to about 20 wt. % of the core.

12. The intravaginal ring device of claim 1, wherein the core is loaded with from about 50 mg to about 1000 mg of one or more aromatase inhibitors.

13. The intravaginal ring device of claim 1, wherein the core is loaded with from about 50 mg to about 400 mg of one or more aromatase inhibitors.

14. The intravaginal ring device of claim 1, wherein the intravaginal ring device is capable of releasing the therapeutic agent for a time period of about 21 days or more.

15. The intravaginal ring device of claim 1, wherein the device is capable of releasing the therapeutic agent for a time period of about 1 month or more.

16. The intravaginal ring device of claim 1, wherein the one or more aromatase inhibitors are released from the device in an amount sufficient to deliver from about 0.5 mg of aromatase inhibitor to about 5 mg of aromatase inhibitor per day.

17. The intravaginal ring device of claim 1, wherein the core polymer matrix comprises one or more plasticizers.

18. The intravaginal ring device of claim 1, wherein the core polymer matrix comprises one or more hydrophilic compounds to control release of the therapeutic agent from the intravaginal ring device.

19. The intravaginal ring device of claim 18, wherein the one or more hydrophilic compounds are present in an amount of from about 1 wt. % to about 60 wt. %.

20. The intravaginal ring device of claim 1, wherein the therapeutic agent is homogenously dispersed within the core polymer matrix.

21. The intravaginal ring device of claim 1, further comprising a first membrane layer positioned adjacent to an outer surface of the core, wherein the first membrane layer comprises a first membrane polymer matrix containing an ethylene vinyl acetate copolymer.

22. The intravaginal ring device of claim 21, wherein the first membrane layer is free of the therapeutic agent.

23. The intravaginal ring device of claim 21, wherein the first membrane layer comprises one or more bisphosphonates.

24. The intravaginal ring device of claim 23, wherein the one or more bisphosphonates are released from the device in an amount sufficient to deliver from about 0.01 mg of bisphosphonate to about 4 mg of bisphosphonate per day.

25. The intravaginal ring device of claim 21, wherein the ethylene vinyl acetate copolymer constitutes an entire polymer content of the first membrane polymer matrix.

26. The intravaginal ring device of claim 21, wherein the first membrane polymer matrix further includes a plasticizer.

27. The intravaginal ring device of claim 21, wherein the first membrane polymer matrix further includes a hydrophobic polymer.

28. The intravaginal ring device of claim 21, wherein the ethylene vinyl acetate copolymer of the first membrane polymer matrix has a melting temperature of from about 40° C. to about 120° C. as determined in accordance with ASTM D3418-15.

29. The intravaginal ring device of claim 21, wherein the ethylene vinyl acetate copolymer of the first membrane polymer matrix has a melt flow index of from about 0.2 to about 100 grams per 10 minutes as determined in accordance with ASTM D1238-20 at a temperature of 190° C. and a load of 2.16 kilograms.

30. The intravaginal ring device of claim 21, wherein the ethylene vinyl acetate copolymer of the first membrane polymer matrix has a vinyl acetate monomer content of from about 10 wt. % to about 50 wt. %.

31. The intravaginal ring device of claim 21, wherein the first membrane polymer matrix comprises one or more hydrophilic compounds to control release of the therapeutic agent from the intravaginal ring device.

32. The intravaginal ring device of claim 31, wherein the one or more hydrophilic compounds are present in an amount of from about 1 wt. % to about 60 wt. %.

33. The intravaginal ring device of claim 31, wherein the one or more hydrophilic compounds include water-soluble particles dispersed within the core polymer matrix.

34. The intravaginal ring device of claim 21, further comprising a second membrane layer positioned adjacent to an outer surface of the first membrane layer, the second membrane layer containing a second membrane polymer matrix.

35. The intravaginal ring device of claim 34, wherein the second membrane layer comprises a second membrane polymer matrix comprising an ethylene vinyl acetate copolymer.

36. The intravaginal ring device of claim 34, wherein the second membrane layer is free of the therapeutic agent.

37. The intravaginal ring device of claim 34, wherein the ethylene vinyl acetate copolymer of the second membrane polymer matrix has a vinyl acetate content that is different from the first membrane polymer matrix and the core polymer matrix.

38. The intravaginal ring device of claim 34, wherein the core, first membrane layer, and/or second membrane layer are formed from a hot melt extrusion process.

39. The intravaginal ring device of claim 34, wherein the core, first membrane layer, and/or second membrane layer are formed from compression molding.

40. A method for prohibiting and/or treating a condition, disease, and/or cosmetic state of a patient, the method comprising intravaginally inserting the device of claim 1 in the patient.
